(19) United States
(12) Patent Application Publication
(10) Pub. No.: US 2022/0118014 A1
(43) Pub. Date: Apr. 21, 2022

(54) PLACENTA-DERIVED ALLOGENEIC CAR-T CELLS AND USES THEREOF

(71) Applicant: Celularity Inc., Florham Park, NJ (US)

(72) Inventors: Robert J. HARIRI, Bernardsville, NJ (US); Kathy KARASIEWICZ-MENDEZ, Hillsborough, NJ (US); Tianjian LI, Princeton, NJ (US)

(73) Assignee: Celularity Inc., Florham Park, NJ (US)

(21) Appl. No.: 17/309,451

(22) PCT Filed: Dec. 2, 2019
(86) PCT No.: PCT/US2019/064074

§ 371 Date: May 27, 2021

Related U.S. Application Data
(60) Provisional application No. 62/878,736, filed on Jul. 25, 2019.
(60) Provisional application No. 62/774,142, filed on Nov. 30, 2018.

(51) Int. Cl. A61K 35/17; A61P 35/02; C07K 14/725; C12N 15/86; C12N 5/073; C12N 5/0783

(52) U.S. Cl. CPC A61K 35/17; A61P 35/02; C12N 5/0636; C12N 15/86; C12N 5/0605; C07K 14/7051

(57) ABSTRACT

The present invention discloses populations of T cells expressing a chimeric antigen receptor (CAR), wherein said T cells are placental T cells derived from cord blood, placental perfusate, or a mixture thereof. Such populations of cells are shown to be improved in a number of aspects over alternative populations of cells such as those derived from peripheral blood mononuclear cell T cells. It also discloses methods of treating cancer, such as a hematologic cancer, e.g., a B cell cancer, or a symptom thereof in a patient in need thereof. These methods comprise administering to the patient an amount of the population of T cells of any one of the invention effective to alleviate the cancer or symptom thereof in the patient.

This application claims priority to U.S. Provisional Patent Application Nos. 62/943,760, filed Dec. 4, 2019, 62/944,349, filed Dec. 5, 2019, and 63/035,432, filed Jun. 5, 2020, the disclosures of which are incorporated herein by reference in their entireties.

### FIELD

The present invention relates, in part, to chimeric antigen receptor (CAR) cells and CAR therapies.

### BACKGROUND

CAR therapies are emerging as a critically important tool against cancer. However, these therapies typically rely on the use of the patient's own cells, e.g., T cells derived from peripheral blood mononuclear cells (PBMCs), as the effector cell population. Because each patient's cells must be harvested, tested, and turned into a CAR therapeutic, CAR therapy is: 1) very expensive; and 2) available at only certain centers willing and/or able to carry out the therapy. These shortcomings result in CAR therapy being largely unavailable to many of the population in need thereof. The subject invention is directed, in part, to creating an allogeneic, off-the-shelf CAR therapy directed to alleviating these and other problems.

Autologous CAR-T therapy has become part of the standard of care for hematological cancer patients. The source of cells of CAR-T therapy comes from the PMBC of the patient. Development of allogeneic CAR-T cell therapy has entered clinical trials which also uses PBMC as the source material. UCB-T cells has different biological properties which makes them more suite to be the source material of allogeneic cell therapy. They have a predominant Tcm and Tnaïve phenotype, display increased proliferative activity, and retain longer telomeres/higher telomerase activity, compared to T cells expanded from PBMCs (Okas, et. al. Journal of Immunotherapy, 2010; Frumento, et. al. Journal of Transplantation, 2013). They have greater immune tolerance to HLA mismatch and impaired allogeneic activation (Barker, et. al. Blood, 2001; Chen, et al. Biology of Blood and Marrow Transplantation, 2006). They can be expanded to clinical scale for therapeutic purposes.

T cell and NK cells are the key cellular mediators of alloreactivity. T cells receptor is the key receptor involved in alloreactivity. T-cell receptor gene inactivation led to reduced alloreactivity. Host NK cells kill donor cells with HLA-mismatched or do not express HLA molecules. One mechanism to evade NK cell killing is through to expression of HLA-E molecule that inhibit NK cell function.

We have developed a unique platform for the use of postpartum human placenta-derived T cells for use in an allogeneic platform for the treatment of hematological and solid cancers. In the present studies we have demonstrated proof of concept with both CD19 CAR-T and CD20 CAR-T cell therapy placental T cells for the treatment of B cell malignancies. Despite placenta-derived T cells (P-T cells) demonstrating greater immune tolerance and impaired allo-responses, we envision, and have demonstrated a T-cell receptor a constant (TRAC) knockout (KO), e.g., a CRISPR-mediated T-cell receptor a constant (TRAC) knockout (KO), step as an additional risk-mitigation strategy to circumvent any potential GvHD stemming from the expression of endogenous T cell receptor on P-T cells. If necessary, these cells can be further genetically modified to NOT express B2M and express a chimeric HLA-E molecule to reduce their alloreactivity/clearance by T/NK cells.

### SUMMARY

The present invention is directed to the use of placenta-derived cells as a source of cells for CAR therapy. These cells include cells isolated from placenta, from placental perfusate and from umbilical cord blood, and combinations thereof. In the present examples, cells from umbilical cord blood and/or from placental perfusate have been used and these placenta-derived cells have been shown to be advantageous over T cells from other cell sources such as those from PBMCs.

Herein, applicants have discovered that placenta-derived cells have a more naïve phenotype with less effector/memory cells than that of PBMCs, representing one advantage of this population. In addition, applicants have demonstrated up to a 3600-fold expansion of the placenta-derived T cells. Based on these discoveries, one aspect of the invention it the use of placenta-derived T cells, e.g. umbilical cord blood-derived T cells or ex vivo expanded umbilical cord blood-derived T cells as a cell type for CAR therapy.

Applicants also have developed methods to do so and shown that such cells can be transduced at high efficiency with an exemplary CAR and readily kill cells expressing the target while not killing cells lacking the target. This killing, or lack thereof, was correlated with expression of effector cytokine expression elicited in response to target-expressing but not target-lacking tumor cells.

Applicants have also demonstrated that placenta-derived T cells are significantly less alloreactive than PBMCs. Thus, in some embodiments, the subject invention teaches the use of placenta-derived cells, e.g., umbilical cord blood-derived cells or expanded umbilical cord blood-derived cells for use in a CAR therapy.

An additional benefit discovered by applicants it that the naïve phenotype of placenta-derived T cells allows for the depletion of Treg cells which might otherwise reduce the effectiveness of the CAR therapy. Such a depletion is not possible/practical for PBMCs due to the expression of CD25 on activated T cells.

In a further effort to create an allogeneic CAR therapy, applicants have knocked out a portion of the TCR, here, the TRAC. Applicants have developed methods to carry out genetic modification of placenta-derived T cells at high efficiency using CRISPR. The use of such a genetic modification is expected to further enhance the allogeneic advantages of placenta-derived T cells. Thus, in some embodiments, the subject invention teaches genetic modification of T cells to reduce alloreactivity such as knocking out a TCR gene, e.g., TRAC.

Although specific CARs have been used in the subject application the advantages of: 1) use of placenta derived T cells; 2) knockout of Tcell genes, e.g., TCR genes such as TRAC; and 3) the combination thereof are expected to be applicable to any CAR and to significantly improve CAR therapy and provide an allogeneic treatment with reduced GVHD.

### BRIEF DESCRIPTION OF THE FIGURES

FIG. 1 shows strategies for circumventing T/NK driven alloreactivity.

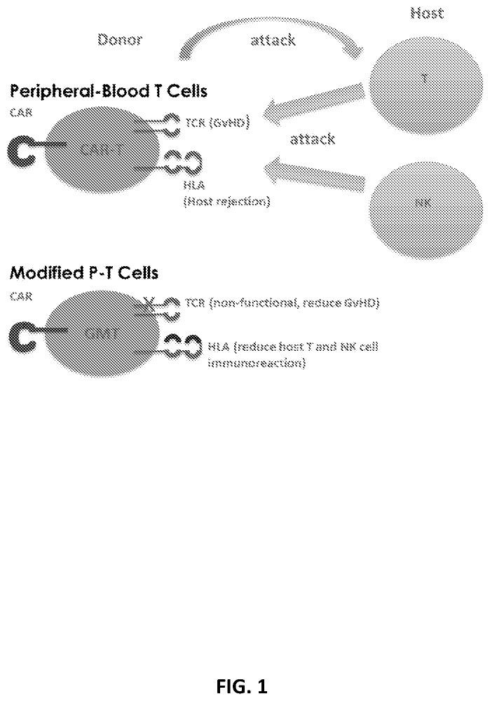

FIG. 2 shows an outline of the process for generating placenta-derived allogeneic CAR-T cells.

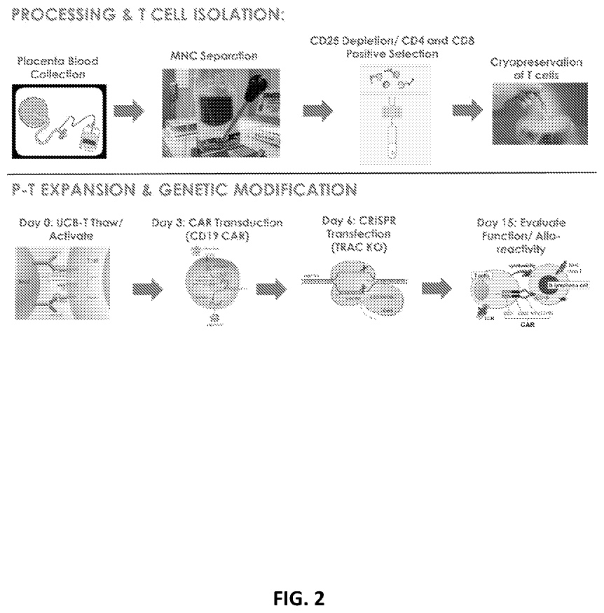

FIG. 3 shows the phenotype of placenta-derived isolated T cells.

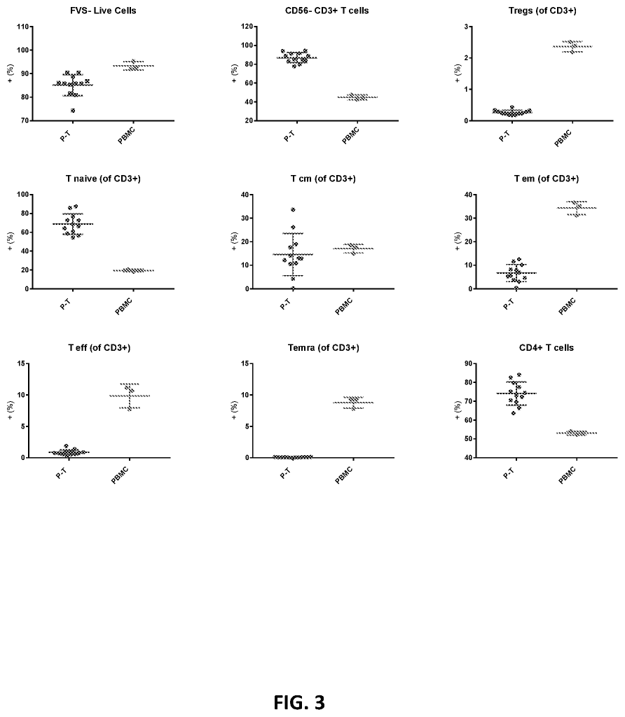

FIG. 4 shows in vitro expansion of placenta-derived T cells at 20 days.

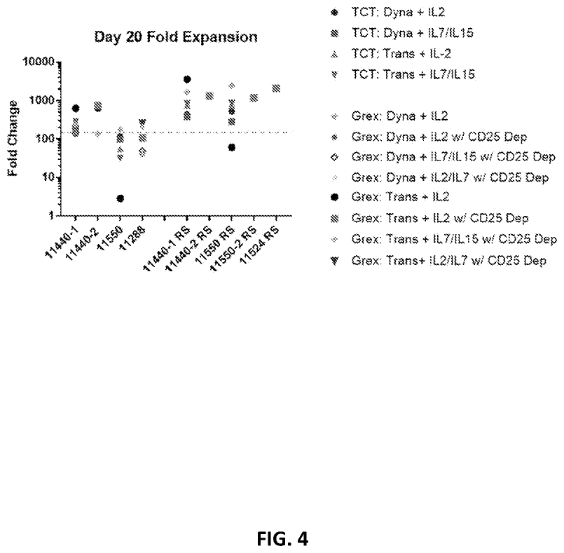

FIG. 5 shows the phenotype of in vitro expanded placenta-derived T cells at 20 days, following restimulation after day 13.

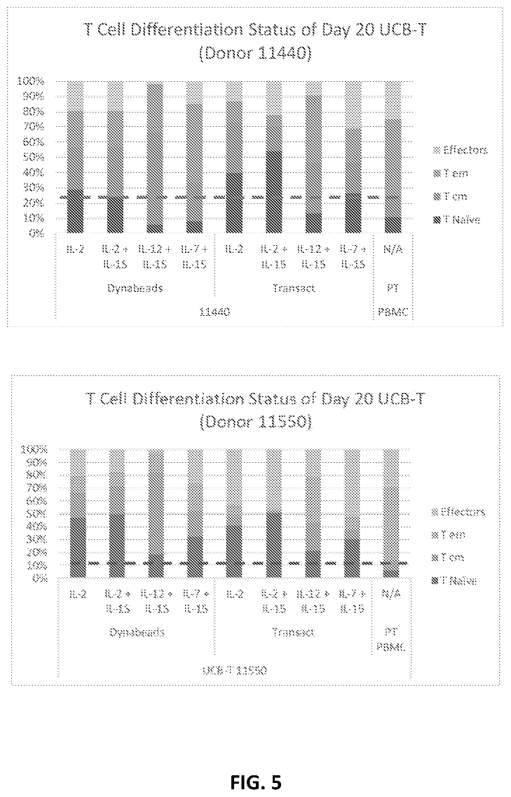

FIG. 6 shows in vitro expansion of CD19 CAR modified placenta-derived T cells at 15 days.

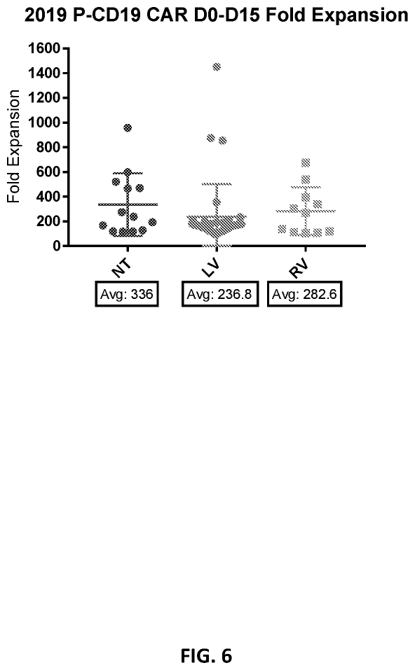

FIG. 7 shows the T cell differentiation status of Day 15 CD19 CAR modified P-T cells.

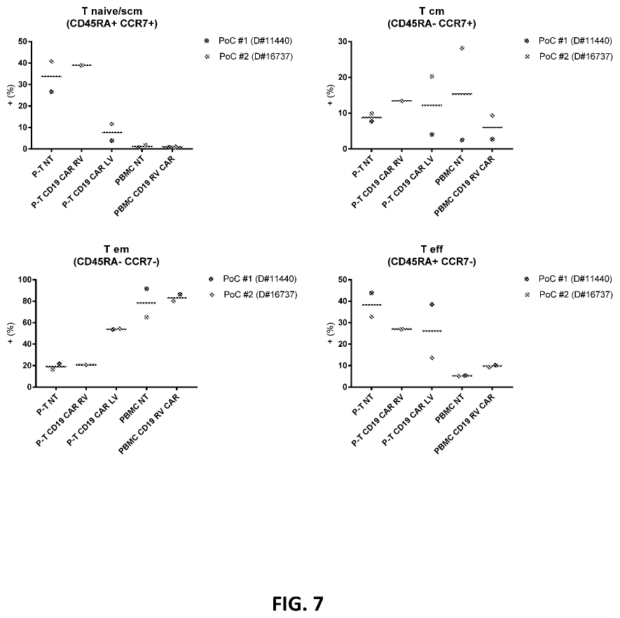

FIG. 8 shows CD57 expression on T effector memory (T em) and T effector (T eff) cells.

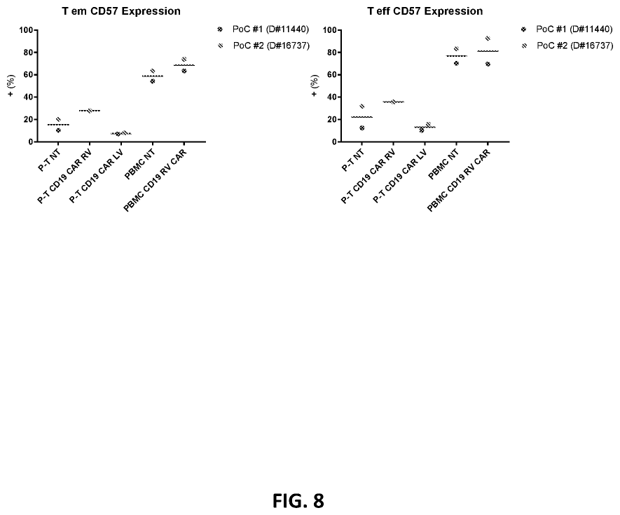

FIG. 9 shows a phenotype analysis of Day 15 CD19 CAR modified P-T cells.

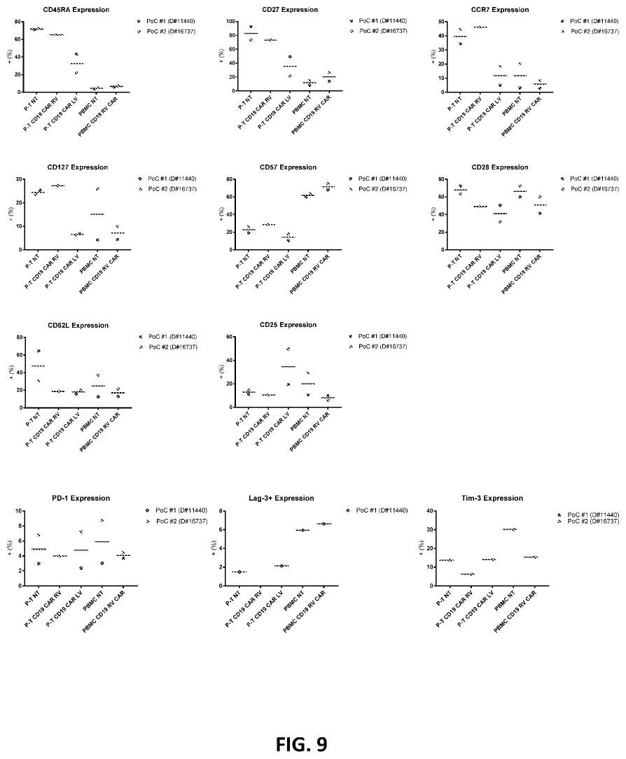

FIG. 10 shows the day 15 CD19 CAR Expression of titrated CD19 CAR viral vectors in P-T cells.

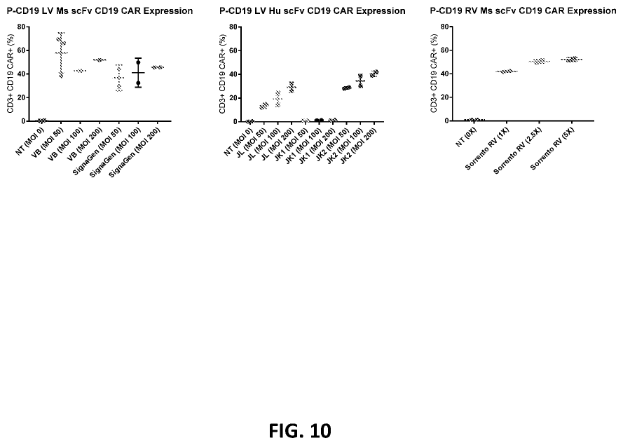

FIG. 11 shows the day 15 P-CD19 CAR phenotype reproduced in multiple P-T preparations from different placenta donors.

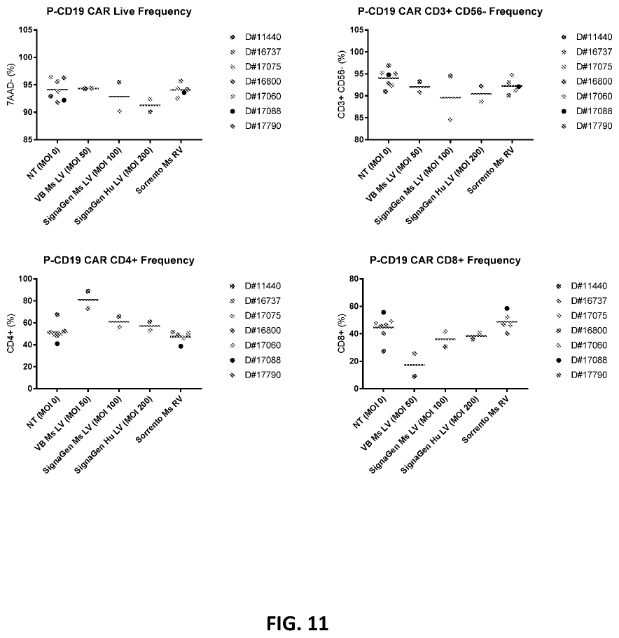

FIG. 12 shows the day 15 CD19 CAR expression reproduced in multiple P-T preparations from different placenta donors.

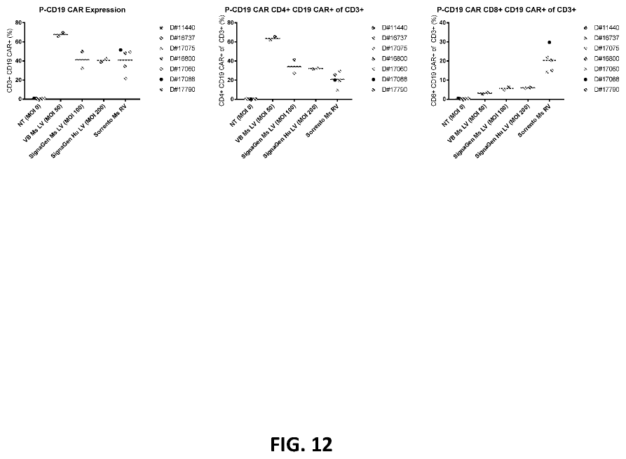

FIG. 13 shows cytotoxicity of Day 14 UCB CD19 CAR-T cells vs. CD19+/CD19− targets (top panels) and cytotoxicity of Day 14 UCB CD20 CAR-T cells vs. CD20+/CD20− targets (bottom panels).

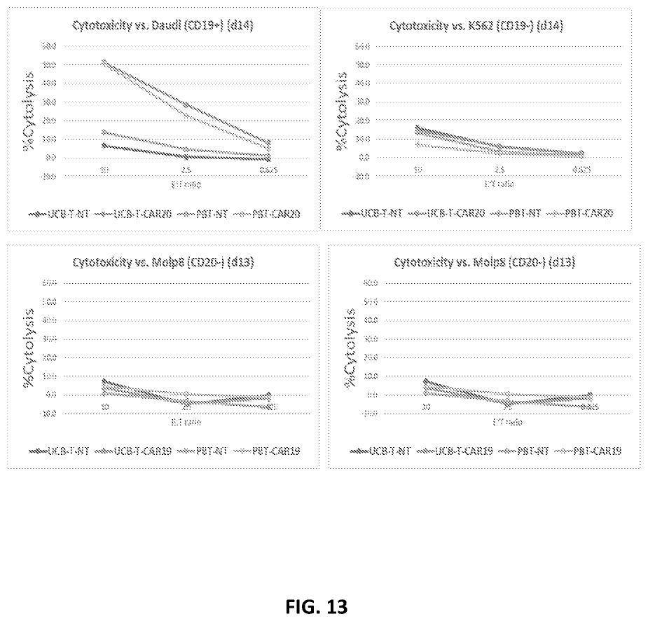

FIG. 14 shows cytokine release of Day 14 UCB CD19 CAR-T cells vs. CD19+/CD19− targets.

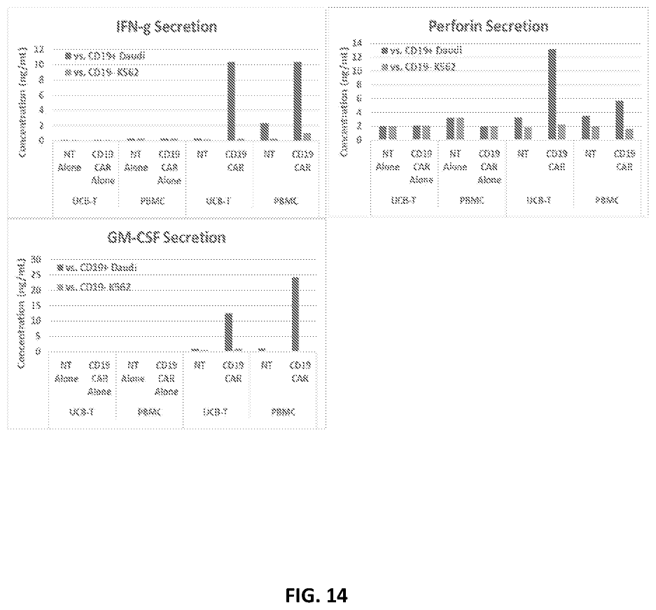

FIG. 15 shows a 4-Hour flow cytotoxicity assay in which Day 15 P-CD19 CAR activity vs. CD19+/− targets is tested.

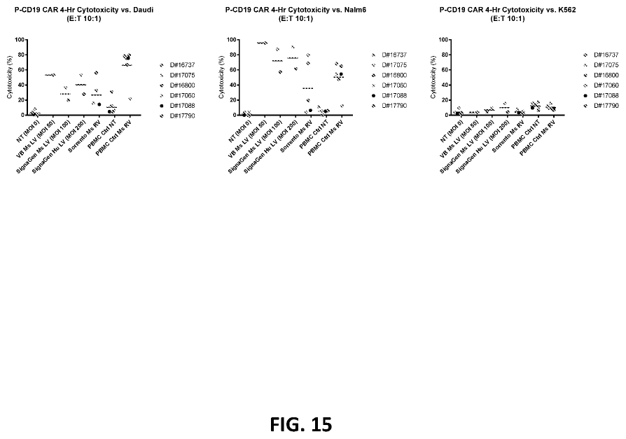

FIG. 16 shows an ACEA kinetic cytotoxicity assay of Day 15 P-CD19 CAR activity vs. CD19+/− targets.

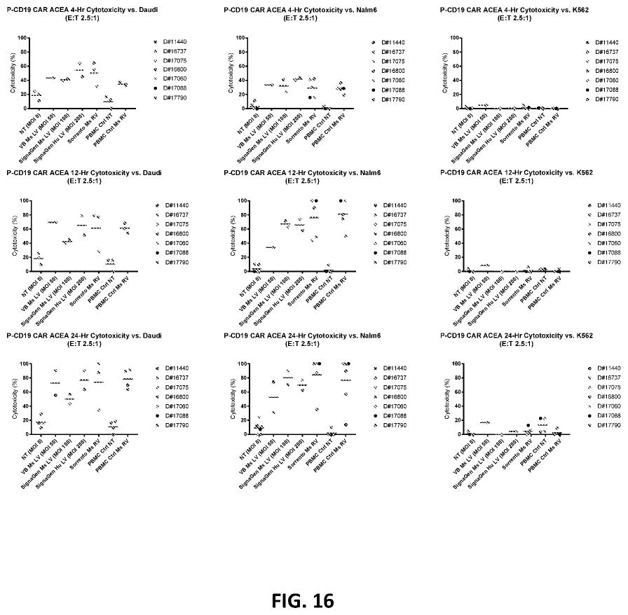

FIG. 17 shows results of a 24-Hour cytokine release assay: Day 15 P-CD19 CAR activity vs. CD19+ Daudi.

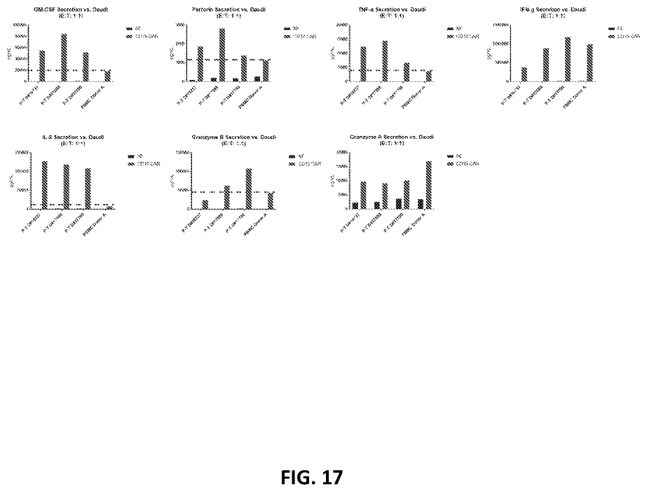

FIG. 18 shows results of a 24-Hour cytokine release assay: Day 15 P-CD19 CAR activity vs. CD19+ Nalm6.

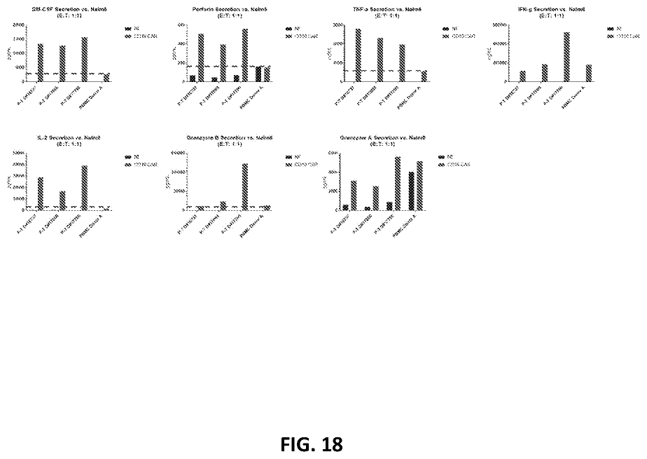

FIG. 19 shows P-CD19 CAR-T activity in a disseminated CD19+ Daudi-Luc mouse model.

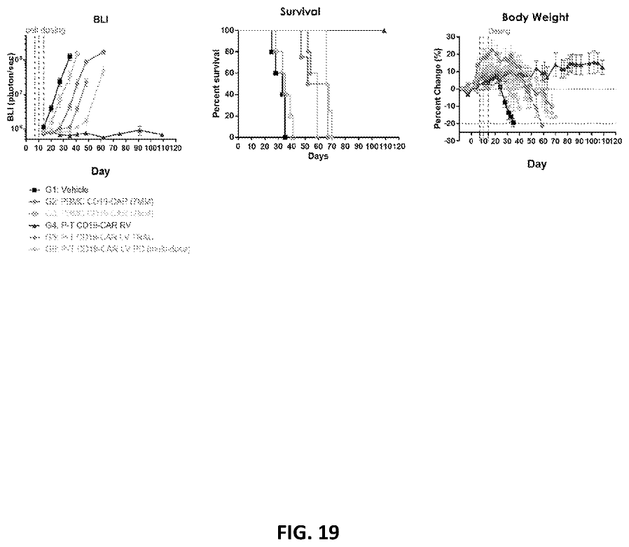

FIG. 20 shows P-CD19 CAR-T activity to tumor cell re-challenge in Daudi-luc disseminated model.

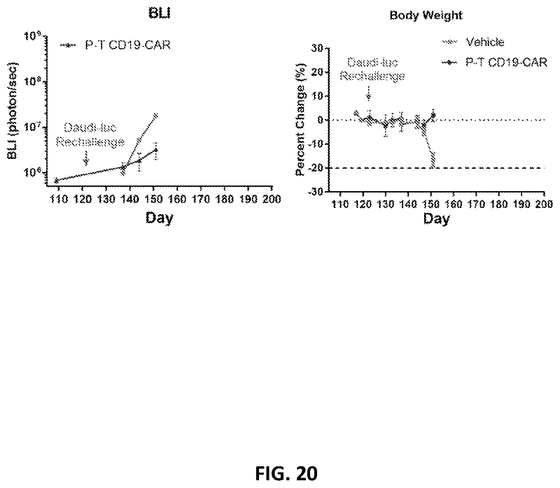

FIG. 21 shows TRAC knockout efficiency in UCB-T cells.

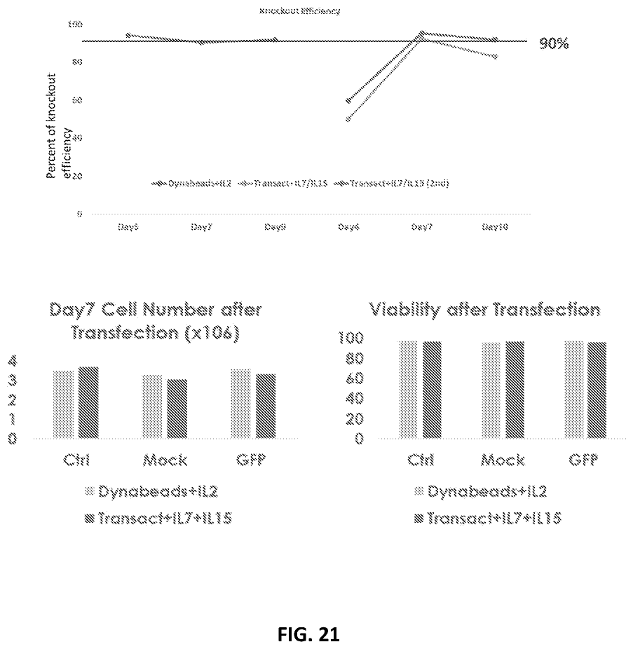

FIG. 22 shows day 15 P-T TRAC KO efficiency using CRISPR.

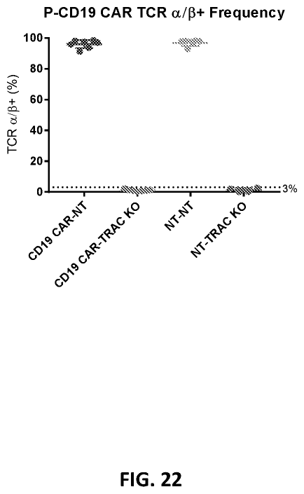

FIG. 23 shows effects of TRAC KO on P-T CD19 CAR expression.

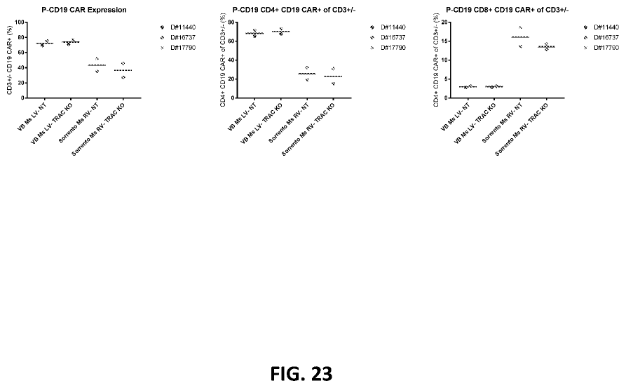

FIG. 24 shows effects of TRAC KO on P-CD19 CAR activity.

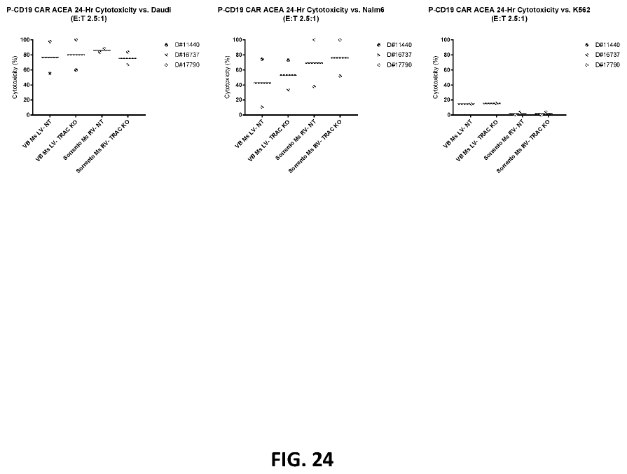

FIG. 25 shows alloreactivity of P-T cells measured by cytotoxicity assay.

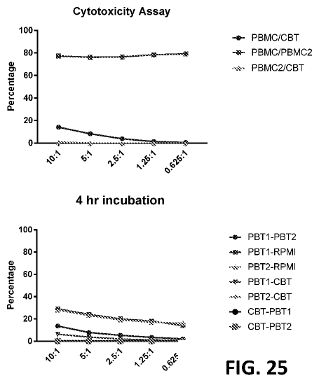

FIG. 26 shows alloreactivity of P-T cells measured by proliferation assay.

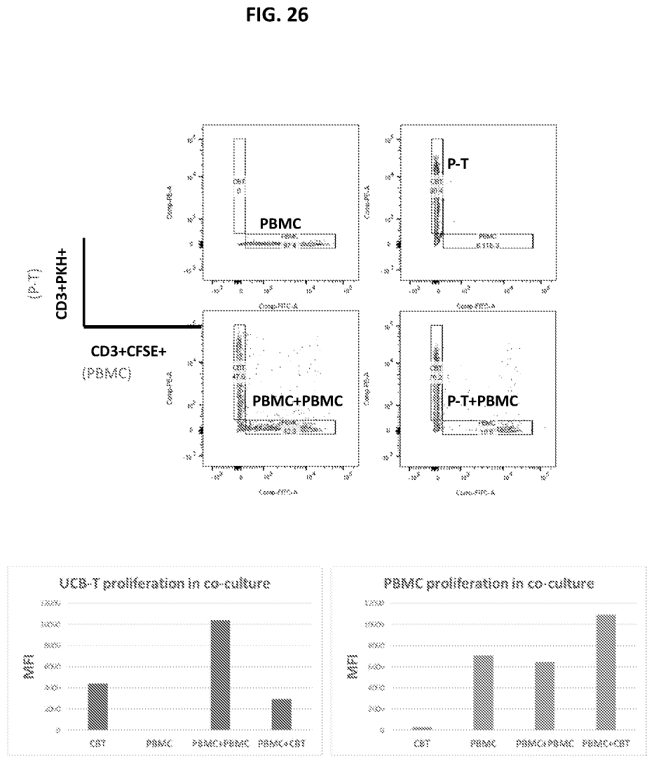

FIG. 27 shows P-T Treg frequency and lack of alloreactivity in an NCG mouse model.

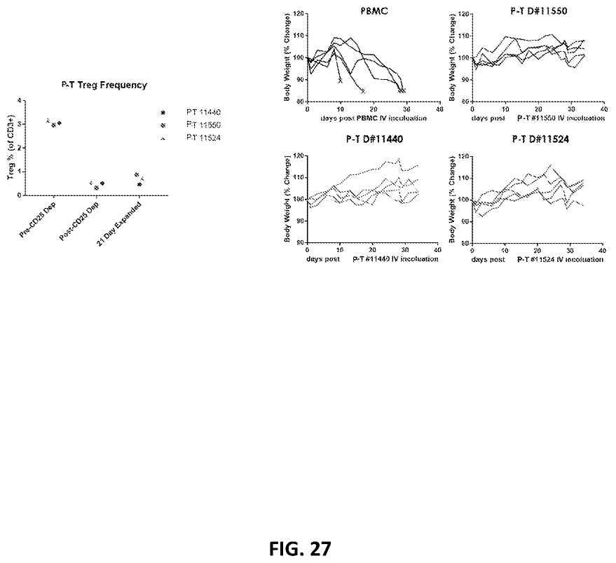

### DETAILED DESCRIPTION

The present invention provides a population of T cells expressing a chimeric antigen receptor (CAR), wherein said T cells are placental T cells. In some embodiments, said placental T cells are cord blood T cells, placental perfusate T cells, or a mixture thereof. In some embodiments, wherein said placental T cells are cord blood T cells. In some embodiments, said placental T cells are a mixture of cord blood T cells and placental perfusate T cells.

In other embodiments, the population of T cells said CAR has been introduced to the cell by transfection. In some embodiments, said CAR has been introduced to the cell by viral transduction. In other embodiments, said CAR has been introduced to the cell by viral transduction with a retroviral vector. In yet other embodiments, said CAR has been introduced to the cell by viral transduction with a lentiviral vector.

These cells have been shown to differ from, e.g., peripheral blood mononuclear derived cells, and indeed to be improved over said cells, in several aspects.

In some embodiments, said population of T cells has a greater percentage of cells expressing CD45RA than a population of peripheral blood mononuclear cell T cells. In other embodiments, said population of T cells has a greater percentage of cells expressing CD27 than a population of peripheral blood mononuclear cell T cells. In other embodiments, said population of T cells has a greater percentage of cells expressing CCR7 than a population of peripheral blood mononuclear cell T cells. In other embodiments, said population of T cells has a greater percentage of cells expressing CD127 than a population of peripheral blood mononuclear cell T cells. In other embodiments, said population of T cells has a lower percentage of cells expressing CD57 than a population of peripheral blood mononuclear cell T cells. In other embodiments, said population of T cells has a greater percentage of cells expressing CD62L than a population of peripheral blood mononuclear cell T cells. In other embodiments, said population of T cells has a lower percentage of cells expressing CD25 than a population of peripheral blood mononuclear cell T cells. In other embodiments, said population of T cells has a greater percentage of cells expressing Lag-3+ than a population of peripheral blood mononuclear cell T cells. In other embodiments, said population of T cells has a lower percentage of cells expressing Tim-3 than a population of peripheral blood mononuclear cell T cells.

In some embodiments, said population of T cells exhibit greater in vitro killing of a cancer cell line than a population of peripheral blood mononuclear cell T cells. In other embodiments, said population of T cells express a greater amount of perforin in an in vitro challenge against a cancer cell line than a population of peripheral blood mononuclear cell T cells. In other embodiments, said population of T cells express a greater amount of GM-CSF in an in vitro challenge against a cancer cell line than a population of peripheral blood mononuclear cell T cells. In other embodiments, said population of T cells express a greater amount of TNF-a in an in vitro challenge against a cancer cell line than a population of peripheral blood mononuclear cell T cells. In other embodiments, said population of T cells express a greater amount of IL-2 in an in vitro challenge against a cancer cell line than a population of peripheral blood mononuclear cell T cells. In other embodiments, said population of T cells express a greater amount of granzyme B in an in vitro challenge against a cancer cell line than a population of peripheral blood mononuclear cell T cells.

In some embodiments, said population of T cells produces increased survival in an in vivo cancer model than a population of peripheral blood mononuclear cell T cells. In other embodiments, said population of T cells produces decreased body weight loss in an in vivo cancer model than a population of peripheral blood mononuclear cell T cells. In other embodiments, said population of T cells produces decreased graft versus host disease (GvHD) in an in vivo cancer model than a population of peripheral blood mononuclear cell T cells.

In other embodiments, said population of peripheral blood mononuclear cell T cells also expresses a said CAR. In other embodiments, said CAR has been introduced to said population of peripheral blood mononuclear cell T cells by transfection. In other embodiments, said CAR has been introduced to said population of peripheral blood mononuclear cell T cells by viral transduction. In other embodiments, said CAR has been introduced to said population of peripheral blood mononuclear cell T cells by viral transduction with a retroviral vector. In other embodiments, said CAR has been introduced to said population of peripheral blood mononuclear cell T cells by viral transduction with a lentiviral vector. In other embodiments, said CAR which has been introduced to said population of peripheral blood mononuclear cell T cells is the same CAR expressed by said population of T cells.

In some embodiments, said population of T cells comprises a further genetic alteration to reduce immunogenicity against a host. In other embodiments, said genetic alteration is a gene knockout. In other embodiments, said gene knockout is a T cell receptor (TCR) knockout. In other embodiments, said gene knockout is a T cell receptor alpha constant (TRAC) knockout. In other embodiments, said further genetic alteration is effected by transfection, retroviral transduction, or lentiviral transduction. In other embodiments, said further genetic alteration is effected by the use of CRISPR, talen, or zn finger technology.

The invention also provides a method of treating cancer or a symptom thereof in a patient in need thereof, the method comprising the step of administering to the patient an amount of the population of T cells of any one of the invention effective to alleviate the cancer or symptom thereof in the patient. In some embodiments, said cancer is a hematologic cancer. In other embodiments, said hematologic cancer is a B cell cancer. In other embodiments, the population of T cells are allogeneic to said patient.

As used herein, “placental perfusate” means perfusion solution that has been passed through at least part of a placenta, e.g., a human placenta, e.g., through the placental vasculature, and includes a plurality of cells collected by the perfusion solution during passage through the placenta.

As used herein, “placental perfusate cells” means nucleated cells, e.g., total nucleated cells, isolated from, or isolatable from, placental perfusate.

As used herein, “tumor cell suppression,” “suppression of tumor cell proliferation,” and the like, includes slowing the growth of a population of tumor cells, e.g., by killing one or more of the tumor cells in said population of tumor cells, for example, by contacting or bringing, e.g., T cells or a T cell population produced using a three-stage method described herein into proximity with the population of tumor cells, e.g., contacting the population of tumor cells with T cells or a T cell population produced using a three-stage method described herein. In certain embodiments, said contacting takes place in vitro or ex vivo. In other embodiments, said contacting takes place in vivo.

As used herein, the term “hematopoietic cells” includes hematopoietic stem cells and hematopoietic progenitor cells.

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “−”, when used to indicate the presence of a particular cellular marker, means that the cellular marker is not detectably present in fluorescence activated cell sorting over an isotype control; or is not detectable above background in quantitative or semi-quantitative RT-PCR.

As used herein, “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect, the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

A CAR that comprises an antigen binding domain (e.g., a scFv, or TCR) that targets a specific tumor maker X, such as those described herein, is also referred to as XCAR. For example, a CAR that comprises an antigen binding domain that targets CD 19 is referred to as CD19CAR.

As used herein, “signaling domain” refers to the functional portion of a protein which acts by transmitting information within the cell to regulate cellular activity via defined signaling pathways by generating second messengers or functioning as effectors by responding to such messengers.

As used herein, “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

As used herein, “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab)₂, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

As used herein, “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof.

As used herein, “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

As used herein, “antibody heavy chain,” refers to the larger of the two types of polypeptide chains present in antibody molecules in their naturally occurring conformations, and which normally determines the class to which the antibody belongs.

As used herein, “antibody light chain,” refers to the smaller of the two types of polypeptide chains present in antibody molecules in their naturally occurring conformations. Kappa (κ) and lambda (λ) light chains refer to the two major antibody light chain isotypes.

As used herein, “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

As used herein, “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

As used herein, “intracellular signaling domain,” refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or FTAM. Examples of ITAM containing primary cytoplasmic signaling sequences include, but are not limited to, those derived from CD3 zeta, common FcR gamma (FCER1G), Fc gamma Rlla, FcR beta (Fc Epsilon Rib), CD3 gamma, CD3 delta, CD3 epsilon, CD79a, CD79b, DAP10, and DAP12.

As used herein, “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO: 18. In one aspect, the “zeta stimulatory domain” or a “CD3-zeta stimulatory domain” is the sequence provided as SEQ ID NO: 20.

As used herein, “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CDl la/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB 1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD 19a, and a ligand that specifically binds with CD83.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-lBB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD 160, B7-H3, and a ligand that specifically binds with CD83, and the like.

The intracellular signaling domain can comprise the entire intracellular portion, or the entire native intracellular signaling domain, of the molecule from which it is derived, or a functional fragment or derivative thereof.

As used herein, “4-IBB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO: 14 or the equivalent residues from a non-human species, e.g., mouse, rodent, monkey, ape and the like.

As used herein, “Immune effector cell,” refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

As used herein, “Immune effector function or immune effector response,” refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

As used herein, “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

As used herein, “autologous” refers to any material derived from the same individual to whom it is later to be re-introduced into the individual.

As used herein, “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

Methods of gene addition/modification are well known in the art and are applicable to the present invention. For example, methods of CAR delivery or gene knockout can be carried out by stable or transient transfection methods or by lentiviral or retroviral transduction. Gene modification can be carried out with these or other methods by the use of, e.g., CRISPR, talen or other such technologies.

### EXAMPLES

### Example 1: Starting Material, MNC Separation, and T Cell Isolation

Starting material Placenta Blood (which includes both Human Umbilical Cord Blood (UCB) and/or Human Placenta Perfusate (HPP)) is collected with informed consent through LifebankUSA. Following collection, the starting materials is enriched for mononuclear cells (MNC) using Hetastarch RBC sedimentation or Ficoll-Paque density gradient cell separation. MNC then undergo a process of positive selection to deplete CD25+ T regulatory T cells (Tregs), followed by positive selection for CD4+ and CD8+ T cells using Militenyi bead cell separation kits. Aliquots of isolated T cells are taken for serology and sterility testing, as well as phenotype analysis, prior to cells being frozen.

The phenotype of isolated P-T cells is distinct from peripheral blood mononuclear cells (PBMCs). P-T cells contain >78% CD3+CD56−T cells and consist mostly of CD3+ CD45RA+ CCR7+ CD27+ naïve T cells with low frequencies of CD3+ CD45RA− CCR7+ CD27+ central memory T cells and CD3+ CD45RA− CCR7− CD27+ effector memory T cells. CD25 depletion significantly reduced the frequency of CD3+ CD4+ CD25+ CD127− Tregs within P-T cells to below 0.5%.

Additional starting material to include, but not yet tested, CD34 Hematopoietic Stem Cells/progenitor-derived Placenta T-cells. Process for expansion and differentiation of progenitors into T cells can take 50-60 days. It is important to note that populations shown below with current protocols have significant populations of CD4+/CD8+ cells are present, however, fully differentiated single positives T cells could readily be selected/enriched for.

Evaluation of Placenta perfusate derived T cells has been completed, but isolation procedure needs to be optimized as current procedure yields low cell numbers, viability, and T cell purity.

### Example 2: T Cell Activation and Expansion

### Non-Modified P-T Cells:

Isolated P-T cells are thawed, undergo CD25-depletion using Miltenyi anti-CD25 beads for removal of CD4+CD25+CD127− Tregs (can be included prior to T cell isolation step), and are activated using anti-CD3/anti-CD28 Dynabeads (1:1 Bead:Cell Ratio) from Invitrogen or using anti-CD3/anti-CD28 nanoparticle Transact (1:100 volumetric dilution) from Miltenyi. Cells are then expanded using 100 IU/mL IL-2, 10 ng/mL IL-7+10 ng/mL IL-15, or 100 IU/mL IL-2+10 ng/mL IL-7. Additional re-stimulations are completed on Days 12-14 and cells are expanded up to Day 21 in Grex vessels to maximize fold expansion.

Non-modified P-T cells can be expanded up to 600-fold with initial stimulation and up to 3,600-fold with re-stimulation (RS) on Day 14 when cultured out to Day 20.

Under various culture conditions, non-modified, 20-Day expanded P-T exhibited an earlier differentiation phenotype compared to post-thaw (PT), non-cultured PBMCs, and consisted mostly of CD3+ CD45RA+ CD62L+ naïve T cells and CD3+ CD45RA− CD62+ central memory T cells, whereas post-thaw, non-cultured PBMCs consisted mostly of more differentiated CD3+ CD45RA−/+CD62L− effector memory and terminal effector T cells. Given the early differentiation status of P-T cells, additional rounds of stimulation should be feasible and significantly increase expansion fold to support “off-the-shelf” manufacture of placenta-derived allogeneic CAR-T, while maintaining a balanced mix of central memory T cells that will persist in the patient, and effector T cells that will immediately target and kill tumor cells.

### CAR Modified P-T Cells:

Isolated T cells (that have undergone CD25-depletion prior to freezing) were thawed and activated using anti-CD3/anti-CD28 nanoparticle Transact (1:100 volumetric dilution) from Miltenyi. Cells were then expanded in Grex vessels using 100 IU/mL IL-2. On Day 3, cells were transduced with either CD19 CAR lentivirus (LV) or retrovirus (RV) on retronectin-coated plates, using the viral pre-spin method. Cells were then culture until Day 15, with media feeds occurring every 2-3 days.

CD19 CAR modified P-T cells can be expanded 237-336-fold following 15 days in culture, without re-stimulation.

Following fifteen days of culture, CD19 CAR modified P-T cells exhibited a distinct T cell differentiation phenotype as compared to CD19 CAR PBMC-derived T cells. P-T cells consisted of a nice mix of CD3+ CD45RA+ CCR7+ naïve/stem cell memory T cells and CD3+ CD45RA+ CCR7− effector T cells, while PBMC-derived CD19 CAR T cells consisted mostly of CD3+ CD45RA− CCR7− effector memory T cells and CD3+ CD45RA+ CCR7− effector T cells. P-T NT (not transduced) and P-T CD19 CAR RV cells consisted of more T naive/scm T cells than P-T CD19 CAR LV cells.

Furthermore, PBMC-derived effector memory T cells (T em) and effector T cells (T eff) expressed significantly higher levels of the exhaustion marker CD57, while P-T cells expression was low.

The greater frequency and mix of effector T cells and naïve/stem cell memory T cells within P-T cells, along with the low CD57 expression, represents a CAR-T product that can efficiently target and kill tumor cells, while maintaining the ability to self-renew and replenish its more differentiated T cell subsets over time.

Overall, Day 15 P-T NT and P-CD19 CART cells expressed high levels of CD45RA, CD27, CCR7, CD127, and CD28, and expressed low levels of the exhaustion marker CD57, and immune checkpoint markers (negative regulators of immune responses) PD-1, Lag-3, and Tim-3.

CD19 CAR transduction efficiency was measured by incubating cells with a CD19 Fc-Fitc reagent and quantifying the percentage CD19 CAR+ cells using flow cytometry. By Day 15, P-T cells expressed CD19 CAR when transduced with all Ms scFv LV or RV (from Vector Builder, SignaGen, or Sorrento) and expressed CD19 CAR when transduced with Hu scFv JK2 and JL sequences, all consisting of the 4-1BB costimulatory domain. P-T cells did not express CD19 CAR when transduced with Hu scFv JK1 sequence, containing the CD28 costimulatory domain. Optimal MOI/concentrations for each CD19 CAR were determined to be: MOI 50 for Vector Builder Ms scFv CD19 CAR LV, MOI 100 for SignaGen Ms scFv CD19 CAR LV, MOI 200 for SignaGen Hu scFv CD19 CAR LV, and 2.5× for Sorrento Ms scFv CD19 CAR RV (calculated titer unknown).

Day 15 P-CD19 CAR T cells exhibited high viability and CD3+ CD56− T cell purity, regardless of viral vector used for transduction. P-T cells transduced with Vector Builder Ms scFv CD19 CAR LV resulted in significantly higher CD4+ T cells, as compared to the same Ms scFv CD19 CAR LV sequence produced by SignaGen. P-T cells transduced with Sorrento's Ms scFv CD19 CAR resulted in the greatest frequency of CD8+ T cells, and a balanced mix of CD4+ and CD8+ T cells.

Using optimized MOIs/concentrations for each CD19 CAR virus type, CD19 CAR expression ranged from 22-70% on Day 15 P-T cells. Vector Builder Ms scFv CD19 CAR LV resulted in the majority of its CD19 CAR expression being expressed on CD4+ T cells, whereas Sorrento's Ms scFv CD19 CAR RV resulted in an equal mix of CD19 CAR expression on CD4+ and CD8+ T cells, and the greatest overall frequency of CD19 CAR expression within CD8+ T cells.

### Example 3: CD19 CAR and CD20 CAR In Vitro Activity

### Cytolytic Activity of Day 15 P-CD19 CAR-T Cells Against Cancer Cell Lines

Activated UCB-T cells were transduced with CD19 CAR retrovirus or lentivirus on Days 2-4 of UCB-T culture using spinoculation. CAR expression were detected using either FITC labeled recombinant CD19-Fc fusion protein or anti-Myc PE antibody, in case the CAR vector contains a Myc tag. UCB-CAR-T activity were assessed using the following two assays.

CD19 CAR transduced UCB-T cells specifically kill CD19+ Daudi cancer targets at levels comparable to PBMC CD19 CART cells, but do not kill CD19− K562 cells.

CD20 CAR transduced UCB-T cells specifically kill CD20+ Daudi cancer targets at levels comparable to PBMC CD20 CAR T cells, but do not kill CD20− Molp8 cells.

CD19 CAR transduced UCB-T cells specifically secrete pro-inflammatory cytokines IFN-g and GM-CSF, and cytolytic effector protein Perforin in response to CD19+ Daudi cancer targets, but not in response to CD19− K562 cells.

In vitro, the functional activity of P-CD19 CART cells also was evaluated vs. CD19+ Burkitt's Lymphoma (Daudi) and CD19+ Acute Lymphoblastic Leukemia (Nalm6) cells lines in a 4-Hour Flow Cytometry-based cytotoxicity assay and a Kinetic ACEA cytotoxicity assay. CD19− K562 cells were included as negative controls to evaluate non-specific killing in both assays.

In both the 4-Hour Flow and the ACEA Kinetic cytotoxicity assays, P-CD19 CAR-T cells specifically killed CD19+ Daudi and Nalm6 cells but did not kill CD19-K562 cells. In the 4-Hour cytotoxicity assay, P-CD19 CAR activity vs. Nalm6 targets was comparable to that of PBMC CD19 CART cells, whereas in the ACEA Kinetic cytotoxicity assay, P-CD19 CAR-T activity was comparable to PBMC CD19 CAR T cells for both Daudi and Nalm6 targets.

Additionally, the In vitro functional activity of P-CD19 CART cells was evaluated vs. CD19+ Burkitt's Lymphoma (Daudi) and CD19+ Acute Lymphoblastic Leukemia (Nalm6) cells lines in a Cytokine Release assay. P-CD19 CAR-T cells were co-culture with CD19+ targets at an E:T ratio of 1:1 for 24-hours, and cell culture supernatants were collected and analyzed for the secretion of various cytokines and effector proteins. Three donors of P-T cells that were transduced with CD19 CAR RV were assessed/compared to PBMC-derived CD19 CAR RV T cells.

Further, P-CD19 CAR-T cells secreted pro-inflammatory cytokines and effector proteins (GM-CSF, Perforin, TNF-a, IFN-g, IL2, Granzyme B, and Granzyme A) in an antigen-specific manner when co-cultured with CD19+ Daudi and Nalm6 targets. Against both CD19+ Daudi and Nalm6 targets, P-CD19 CAR T cells secreted higher concentrations of GM-CSF, Perforin, TNF-a, Granzyme B, and especially IL2 as compared to their PBMC-derived count parts. The significantly higher secretion of IL2 is indicative of a less differentiated, more stem-like population, and can promote greater T cell expansion, enhanced T cell function, and survival.

### Example 4: P-CD19 CAR-T In Vivo Activity

In vivo, the anti-tumor activity of P-CD19 CART cells was assessed using a disseminated lymphoma xenograft model in NSG mice. Luciferase expressing Daudi cells (3×106) were intravenously (IV) injected on Day 0, followed by IV injection of P-CD19 CAR T cells. P-T cells were dosed according to CD8+ CD19 CAR+ frequencies outlined in table 1 (P-T: RV: one dose of 14×106 on Day 7; LV: one dose of 20×106 on Day 7 or three doses of 20×106 on Days 7, 10, and 14). Bioluminescence Imaging (BLI) and survival were used as primary study endpoints.

| | | | | | Pre-Freeze | | |
|---|---|---|---|---|---|---|---|
| | | | | | % CD19 | CD19 | |
| | | # of | Dosing | T Cell | CAR+ CD8+ | CAR+ CD8+ | |
| Group | Treatment | Animals | Schedule | Dosage | (of CD3+) | Cell Dose | ROA |
| 1 | Vehicle (PBS) | 5 | Day 7 | N/A | N/A | N/A | IV |
| 2 | PBMC CD19- | 5 | Day 7 | 7 MM | 30.59% | 2.14 MM | |
| 3 | CAR RV | 5 | Day 7 | 2 MM | 30.59% | 0.612 MM | |
| 4 | P-T CD19- | 5 | Day 7 | 14 MM | 15.01% | 2.1 MM | |
| | CAR RV | | | | | | |
| 5 | P-T CD19-CAR | 5 | Day 7 | 20 MM | 3.11% | 0.622 MM | |
| | LV-TRAC KO | | | | | | |
| 6 | P-T CD19-CAR | 5 | Days 7, | 20 MM | 3.50% | 3 × 0.7 MM = | |
| | LV (multi-dose) | | 10, 14 | | | 2.1 MM | |

P-CD19 CART cells were well tolerated and safe in this mouse model, even at three doses of 20×106 of non-TRAC modified T cells. All P-CD19 CART cells significantly reduced tumor burden and improved survival. At four weeks after treatment, the vehicle group had a 100% mortality rate, while all animals from P-CD19 CAR T-treated group (N=5) remained alive without clinical symptoms including weight loss. P-CD19 CAR LV treated groups managed tumor burden as well as the PBMC CD19 CAR (7MM) treated group. Multi-dosing (3×) with P-CD19 CAR LV cells demonstrated improvement over a single dose and exhibited slightly better tumor management and survival than by the 7MM PBMC CD19-CAR RV treated group (both dosed at a total of 2.1MM CD19-CAR+ CD8+ T cells). Notably, the single dose of P-CD19 CAR LV cells (0.6MM CD19-CAR+ CD8+ T cells) reduced tumor burden and improved survival better than the 2MM PBMC CD19 CAR RV treated group (also 0.6MM CD19-CAR+ CD8+ T cells). Remarkably, the P-CD19 CAR RV treated mice out-performed all treatment groups and eradicated tumor cells with 100% survival out to Day 109. The less differentiated T cell phenotype, along with the presence of both naïve/scm and effector T cells, a good mix of CD4+ and CD8+ T cells, greater CD8+ CD19 CAR+ expression, and greater cytokine secretion (especially IL2 to support T cell function/survival), all described herein, are believed to collectively contribute to the greater efficacy and enhanced survival observed in vivo with P-CD19 CAR T cells, especially the P-CD19 CAR RV T cells.

The surviving mice from the P-CD19 CAR RV treated group were then re-challenged with additional Daudi tumor cells. On Day 122, luciferase expressing Daudi cells (3×106) were intravenously (IV) injected into the P-CD19 CAR RV treated surviving mice, as well as age-matched (6-month-old) naïve NSG mice, to serve as the new vehicle control group.

This study is still ongoing, but at Day 151 (28 days post re-challenge) we have observed significantly lower BLI (tumor burden) and no clinical symptoms (weight loss) within the P-CD19 CAR RV re-challenge group, whereas we have detected increases in BLI and weight loss with the Vehicle control group. BLI, body weight, and survival will continue to be monitored and is expected to remain improved.

### Example 5: T-Cell Receptor (TRAC) Knock-Out in UCB-T Cells

TRAC was targeted using guide RNA (gRNA) against the first exon of TRAC locus. Chemically modified RNA forms of Cas9 and gRNA were transfected into P-T cells at day 6-8 of P-T culture via Nucleofection (Lonza). Gene modification efficiency were monitored by flow cytometry using antibody against TCR_{□□□} or CD3.

In three separate experiments, TRAC knockout efficiency was measured 3 days after transfection. The date on the x-axis indicates the time of transfection. Over 90% TRAC gene knockout were achieved regardless of the method of P-T activation and culture conditions (Dynabeads with IL2 or Transact with IL7 and IL15). B. Cell proliferation and viability was minimally impacted by the CRISPR process. There is no significant change of cell proliferation and viability among different groups.

Additionally, when P-T cells were transduced with CD19 CAR LV or RV on Day 3, followed by transfection and TRAC KO using CRISPR on Day 6, Day 15 P-T NT-TRAC KO and P-CD19 CAR-TRAC KO cells exhibited >97% TRAC KO efficiency.

Furthermore, TRAC KO did not result in any significant changes in CD19 CAR expression or in vitro cytolytic activity vs. CD19+ Daudi and Nalm6 targets in P-T cells.

### Example 6: Alloreactivity of UBC-T Cells Measured in In Vitro Assays

Two independent assays were used to measure alloreactivity of PMBCs against P-T cells, or P-T cells against PBMCs. In the first one, alloreactivity was measured as killing activity of cells from one donor to against another in a 4-hour co-culture. Target cells were labeled with PKH26 and cytotoxicity was expressed as percentage of dead target cells over total target cells. In the second one, alloreactivity was measured as preferential proliferation of T cells of one donor when co-cultured with another. Cells from two donors are labeled with different dyes (CFSE and PKH26) and co-cultured at 1:1 ratio for 4 days. Dilution of the dye is indicative of cell proliferation and can be expressed as a decrease of percentage of cells with high intensity or change of mean fluorescent intensity.

In two separate experiments, PBMCs or PBMC derived T cells were co-cultured with P-T cells. PBMCs from one donor killed PBMCs from another donor with high efficiency. But PBMCs did not kill P-T cells (CBT). In a separate experiment, PMBC derived T cells (PBT) killed cancer cell line RPMI8226 (RPMI) with high efficiency. But they had minimal activity at killing P-T cells (CBT). P-T cells did not kill PBMC derived T cells either.

P-T cells and control PBMCs were labeled with PKH26 and PBMCs are labeled with CFSE. CFSE labeled PBMC, PHK26 labeled P-T (CBT), and mixed culture of PBMC labeled with either CFSE or PKH26 served as controls. There is lower percentage of PKH26-hi P-T (CBT) cells compared to P-T only culture, indicative of preferential proliferation of P-T cell in co-culture with PBMCs.

Consistent with this result, the MFI of P-T cells also dropped in co-culture with PBMCs compared to P-T cell only and PBMC with PBMC control indicative of better proliferation. In contrast, the MFI of PBMCs in co-culture increased compared to PBMCs only or PBMC with PBMC culture.

### Example 7: Alloreactivity of P-T Cells in Animal Models

Alloreactivity (xeno-alloreactivity) of non-modified, 21-Day expanded P-T cells was tested in an NCG mouse model of GvHD. In this model, PBMC causes GvHD which can be measured as weight loss. 30 million of CD25 depleted P-T cell from three donors and control PMBCs were injected into NCG mice via IV route. Animal weight were monitored over time.

Body weight change of animals was expressed as percentage of body weight on the day of cell injection. Each line represents one mouse. All five animals in the PBMC group lost weight over the course of 28 days and had to be sacrificed. None in the P-T group had significant weight loss and did induce xeno-GvHD. P-T cells were CD25-depleted prior to expansion to remove Tregs, so lack of GvHD is not attributed to CD4+ CD25+ CD127-FoxP3+ immune regulatory T cells. Additional GvHD studies are underway to evaluate the alloreactive of P-CD19 CAR-T and P-CD19 CAR− TRAC KO T cells.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

1. A population of T cells expressing a chimeric antigen receptor (CAR), wherein said T cells are placental T cells.

2. The population of T cells of claim 1, wherein said placental T cells are cord blood T cells, placental perfusate T cells, or a mixture thereof.

3. The population of T cells of claim 1, wherein said placental T cells are cord blood T cells.

4. The population of T cells of claim 1, wherein said placental T cells are a mixture of cord blood T cells and placental perfusate T cells.

5. The population of T cells of any one of claims 1-4, wherein said CAR has been introduced to the cell by transfection.

6. The population of T cells of any one of claims 1-4, wherein said CAR has been introduced to the cell by viral transduction.

7. The population of T cells of claim 6, wherein said CAR has been introduced to the cell by viral transduction with a retroviral vector.

8. The population of T cells of claim 6, wherein said CAR has been introduced to the cell by viral transduction with a lentiviral vector.

9. The population of T cells of any one of claims 1-8, wherein said population of T cells has a greater percentage of cells expressing CD45RA than a population of peripheral blood mononuclear cell T cells.

10. The population of T cells of any one of claims 1-9, wherein said population of T cells has a greater percentage of cells expressing CD27 than a population of peripheral blood mononuclear cell T cells.

11. The population of T cells of any one of claims 1-10, wherein said population of T cells has a greater percentage of cells expressing CCR7 than a population of peripheral blood mononuclear cell T cells.

12. The population of T cells of any one of claims 1-11, wherein said population of T cells has a greater percentage of cells expressing CD127 than a population of peripheral blood mononuclear cell T cells.

13. The population of T cells of any one of claims 1-12, wherein said population of T cells has a lower percentage of cells expressing CD57 than a population of peripheral blood mononuclear cell T cells.

14. The population of T cells of any one of claims 1-13, wherein said population of T cells has a greater percentage of cells expressing CD62L than a population of peripheral blood mononuclear cell T cells.

15. The population of T cells of any one of claims 1-14, wherein said population of T cells has a lower percentage of cells expressing CD25 than a population of peripheral blood mononuclear cell T cells.

16. The population of T cells of any one of claims 1-15, wherein said population of T cells has a greater percentage of cells expressing Lag-3+ than a population of peripheral blood mononuclear cell T cells.

17. The population of T cells of any one of claims 1-16, wherein said population of T cells has a lower percentage of cells expressing Tim-3 than a population of peripheral blood mononuclear cell T cells.

18. The population of T cells of any one of claims 1-17, wherein said population of T cells exhibit greater in vitro killing of a cancer cell line than a population of peripheral blood mononuclear cell T cells.

19. The population of T cells of any one of claims 1-18, wherein said population of T cells express a greater amount of perforin in an in vitro challenge against a cancer cell line than a population of peripheral blood mononuclear cell T cells.

20. The population of T cells of any one of claims 1-19, wherein said population of T cells express a greater amount of GM-CSF in an in vitro challenge against a cancer cell line than a population of peripheral blood mononuclear cell T cells.

21. The population of T cells of any one of claims 1-20, wherein said population of T cells express a greater amount of TNF-a in an in vitro challenge against a cancer cell line than a population of peripheral blood mononuclear cell T cells.

22. The population of T cells of any one of claims 1-21, wherein said population of T cells express a greater amount of IL-2 in an in vitro challenge against a cancer cell line than a population of peripheral blood mononuclear cell T cells.

23. The population of T cells of any one of claims 1-22, wherein said population of T cells express a greater amount of granzyme B in an in vitro challenge against a cancer cell line than a population of peripheral blood mononuclear cell T cells.

24. The population of T cells of any one of claims 1-23, wherein said population of T cells produces increased survival in an in vivo cancer model than a population of peripheral blood mononuclear cell T cells.

25. The population of T cells of any one of claims 1-24, wherein said population of T cells produces decreased body weight loss in an in vivo cancer model than a population of peripheral blood mononuclear cell T cells.

26. The population of T cells of any one of claims 1-25, wherein said population of T cells produces decreased graft versus host disease (GvHD) in an in vivo cancer model than a population of peripheral blood mononuclear cell T cells.

27. The population of T cells of any one of claims 9-26, wherein said population of peripheral blood mononuclear cell T cells also expresses a said CAR.

28. The population of T cells of claim 27, wherein said CAR has been introduced to said population of peripheral blood mononuclear cell T cells by transfection.

29. The population of T cells of claim 27, wherein said CAR has been introduced to said population of peripheral blood mononuclear cell T cells by viral transduction.

30. The population of T cells of claim 29, wherein said CAR has been introduced to said population of peripheral blood mononuclear cell T cells by viral transduction with a retroviral vector.

31. The population of T cells of claim 29, wherein said CAR has been introduced to said population of peripheral blood mononuclear cell T cells by viral transduction with a lentiviral vector.

32. The population of T cells any one of claims 1-31, wherein said CAR which has been introduced to said population of peripheral blood mononuclear cell T cells is the same CAR expressed by said population of T cells.

33. The population of T cells any one of claims 1-32, wherein said population of T cells comprises a further genetic alteration to reduce immunogenicity against a host.

34. The population of T cells claim 33, wherein said genetic alteration is a gene knockout.

35. The population of T cells claim 34, wherein said gene knockout is a T cell receptor (TCR) knockout.

36. The population of T cells claim 34, wherein said gene knockout is a T cell receptor alpha constant (TRAC) knockout.

37. The population of T cells any one of claims 33-36, wherein said further genetic alteration is effected by transfection, retroviral transduction, or lentiviral transduction.

38. The population of T cells any one of claims 33-36, wherein said further genetic alteration is effected by the use of CRISPR, talen, or zn finger technology.

39. A method of treating cancer or a symptom thereof in a patient in need thereof, the method comprising the step of administering to the patient an amount of the population of T cells of any one of claims 1-38 effective to alleviate the cancer or symptom thereof in the patient.

40. The method of claim 39, wherein said cancer is a hematologic cancer.

41. The method of claim 40, wherein said hematologic cancer is a B cell cancer.

42. The method of any one of claims 39-41, wherein the population of T cells are allogeneic to said patient.
